(19) United States
(12) Patent Application Publication
(10) Pub. No.: US 2026/0026699 A1
(43) Pub. Date: Jan. 29, 2026

(54) HEART RATE DETECTION SYSTEM, HEART RATE DETECTION METHOD, AND PROGRAM

(71) Applicant: SEKISUI HOUSE, LTD., Osaka-shi, Osaka (JP)

(72) Inventors: Buntoku MORI, Osaka-shi, Osaka (JP); Masae NAKAMOTO, Osaka-shi, Osaka (JP); Akihiro FUKUSHIMA, Osaka-shi, Osaka (JP)

(21) Appl. No.: 18/997,097

(22) PCT Filed: Apr. 4, 2023
(86) PCT No.: PCT/JP2023/013973

§ 371 Date: Jan. 20, 2025

(30) Foreign Application Priority Data
Jul. 21, 2022 (JP) 2022-116727

(51) Int. Cl. A61B 5/024; A61B 5/0507

(52) U.S. Cl. CPC A61B 5/024; A61B 5/0507

(57) ABSTRACT

To accurately measure a heart rate of a person to be measured, provided is a heart rate detection system (1) including: a heartbeat data acquisition module (300) which acquires heartbeat data indicating a heartbeat of a person to be measured in each of a plurality of time windows based on a Doppler signal; an FFT module (32) which converts respective pieces of the heartbeat data into frequency spectra; a peak identification module (33) which identifies peaks included in each of the frequency spectra; a peak tracking module (34) which determines whether corresponding peaks are present in the frequency spectra relating to a predetermined number of consecutive time windows, the predetermined number being three or more; and a heart rate generation module (301) which generates a heart rate of the person to be measured based on a result of the determination.

### TECHNICAL FIELD

The present invention relates to a heart rate detection system, a heart rate measurement method, and a program, and more particularly, to a system for detecting a heart rate of a person to be measured based on a Doppler signal.

### BACKGROUND ART

Various systems for measuring a heart rate of a person to be measured have been considered. In a related-art system in which an electrode is brought into contact with a person to be measured to measure a cardiac potential, a heavy load is imposed on the person to be measured, and hence, as described in Patent Literature 1, a method of measuring a heart rate in a non-contact manner through use of a microwave Doppler sensor is considered as promising. With the microwave Doppler sensor, the heart rate can be acquired by measuring movement of a body surface of the person to be measured or movement within a body of the person to be measured.

In a heartbeat detection system as described in Patent Literature 2, an estimated value of the heart rate is obtained from a maximum peak of a heartbeat spectrum.

### CITATION LIST

### Patent Literature

[PTL 1] JP 2017-134795 A
[PTL 2] JP 2019-129996 A

### SUMMARY OF INVENTION

### Technical Problem

However, a heartbeat spectrum actually obtained from a person to be measured includes a large number of noise components, and a maximum peak thereof does not always indicate a heart rate of the person to be measured.

The present invention has been made in view of the above-mentioned problem, and has an object to provide a heart rate detection system, a heart rate detection method, and a program with which a heart rate of a person to be measured can be accurately measured.

### Solution to Problem

(1) According to one embodiment of the present invention, there is provided a heart rate detection system including: acquisition means for acquiring heartbeat data indicating a heartbeat of a person to be measured in each of a plurality of time windows based on a Doppler signal; conversion means for converting respective pieces of the heartbeat data into frequency spectra; peak identification means for identifying peaks included in each of the frequency spectra; peak tracking means for determining whether corresponding peaks are present in the frequency spectra relating to a predetermined number of consecutive time windows, the predetermined number being three or more; and heart rate generation means for generating a heart rate of the person to be measured based on a result of the determination.
(2) In the heart rate detection system according to Item (1), the heart rate generation means may be configured to generate, when the corresponding peaks are present in the frequency spectra relating to the predetermined number of consecutive time windows, the heart rate of the person to be measured based on at least some of the corresponding peaks.
(3) In the heart rate detection system according to Item (1) or (2), the peak tracking means may be configured to determine that the corresponding peaks are present when values of frequencies of peaks relating to temporally adjacent time windows are within a predetermined range.
(4) In the heart rate detection system according to any one of Items (1) to (3), the peak tracking means may be configured to determine that the corresponding peaks are present when all values of frequencies of peaks relating to the predetermined number of consecutive time windows are within a predetermined range.
(5) The heart rate detection system according to any one of Items (1) to (4) may further include amplitude ratio calculation means for calculating an amplitude ratio of a peak included in each of the frequency spectra to another peak. The heart rate generation means may be configured to generate the heart rate of the person to be measured further based on the amplitude ratio.
(6) In the heart rate detection system according to Item (5), the heart rate generation means may be configured to generate the heart rate of the person to be measured based on only a peak the amplitude ratio of which is equal to or larger than a predetermined value.
(7) In the heart rate detection system according to any one of Items (1) to (6), the heart rate generation means may include means for calculating temporary heart rates corresponding to the respective time windows. The heart rate generation means may be configured to avoid generating the heart rate of the person to be measured when the number of temporary heart rates that satisfy a predetermined criterion among the temporary heart rates calculated during a predetermined period is smaller than a predetermined number.
(8) According to one embodiment of the present invention, there is provided a heart rate detection method including the steps of: acquiring heartbeat data indicating a heartbeat of a person to be measured in each of a plurality of time windows based on a Doppler signal; converting respective pieces of the heartbeat data into frequency spectra; identifying peaks included in each of the frequency spectra; determining whether corresponding peaks are present in the frequency spectra relating to a predetermined number of consecutive time windows, the predetermined number being three or more; and generating a heart rate of the person to be measured based on a result of the determination.
(9) According to one embodiment of the present invention, there is provided a program for causing a computer to execute the steps of: acquiring heartbeat data indicating a heartbeat of a person to be measured in each of a plurality of time windows based on a Doppler signal; converting respective pieces of the heartbeat data frequency spectra; identifying peaks included in each of the frequency spectra; determining whether corresponding peaks are present in the frequency spectra relating to a predetermined number of consecutive time windows, the predetermined number being three or more; and generating a heart rate of the person to be measured based on a result of the determination. The program may be stored in a computer-readable information storage medium.

### Advantageous Effects of Invention

According to the present invention, the heart rate of the person to be measured can be accurately measured.

### BRIEF DESCRIPTION OF DRAWINGS

FIG. 1 is a configuration diagram of a heart rate detection system according to an embodiment of the present invention.

FIG. 2 is a functional block diagram of a signal processing device in the embodiment of the present invention.

FIG. 3 is a diagram for illustrating processing performed by a heartbeat data cut-out module.

FIG. 4 is a diagram for illustrating details of a second filter module.

FIGS. 5 are graphs for showing output of an FFT module, in which FIG. 5(a) shows a frequency spectrum at a time “t” and

FIG. 5(b) shows a frequency spectrum at a time t+1.

FIG. 6 is a table for schematically showing stored information of a peak storage unit.

FIG. 7 is a flow chart for illustrating processing performed by a peak tracking module.

FIG. 8 is a table for schematically showing stored information of a temporary heart rate storage unit.

### DESCRIPTION OF EMBODIMENTS

Now, an embodiment of the present invention is described in detail with reference to the accompanying drawings.

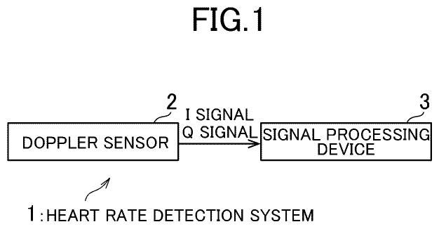

FIG. 1 is a configuration diagram of a heart rate detection system according to the embodiment of the present invention. As illustrated in the figure, the heart rate detection system 1 includes a Doppler sensor 2 and a signal processing device 3. The heart rate detection system 1 is set in a house, for example, and detects, for example, a heart rate of a person to be measured who is sleeping. The Doppler sensor 2 is installed toward a heart of the person to be measured in the vicinity of a bed, for example. A microwave is emitted from the Doppler sensor 2, and the wave reflected by a portion around the heart of the person to be measured is received by the Doppler sensor 2. With the Doppler effect, the reflected wave is shifted in frequency, and a heart rate of the person to be measured can be obtained by observing the reflected wave. The reflected wave is detected as a Doppler signal including an I signal being an in-phase component with respect to the transmitted wave and a Q signal being a quadrature component with respect to the transmitted wave, and is output to the signal processing device 3 in a digital form. The Doppler signal input to the signal processing device 3 is time-series data, and indicates an amplitude at each time (I component and Q component).

The signal processing device 3 may be formed of a publicly-known computer including, for example, a CPU, a memory, an input device, and a display, and the signal processing device 3 generates the heart rate of the person to be measured based on the Doppler signal output from the Doppler sensor 2.

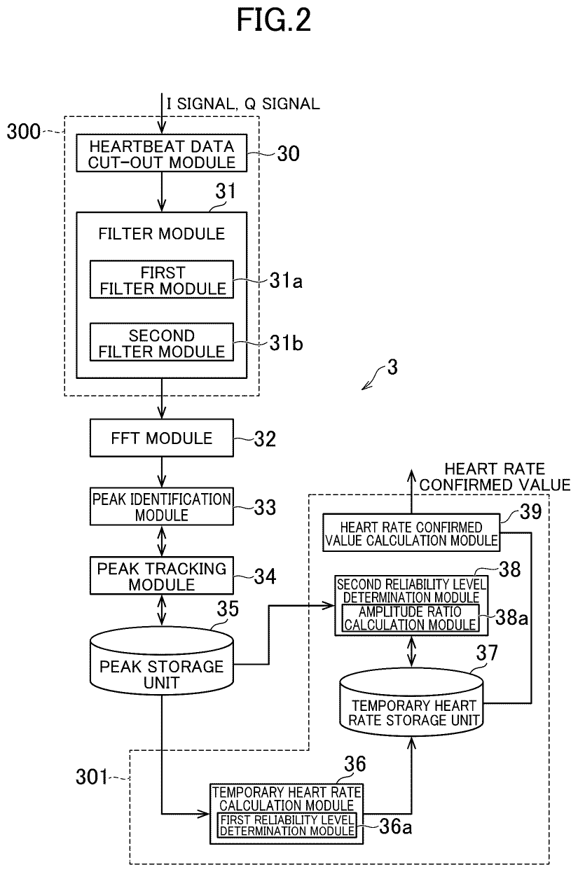

FIG. 2 is a functional block diagram of the signal processing device 3 in the embodiment of the present invention. As illustrated in the figure, the signal processing device 3 includes a heartbeat data acquisition module 300, an FFT module 32, a peak identification module 33, a peak tracking module 34, a peak storage unit 35, and a heart rate generation module 301. Those functional blocks are implemented when a signal processing program is executed by the signal processing device 3, which is the computer. The signal processing program may be stored in one of various computer-readable information storage media such as a semiconductor memory, and may be loaded from the medium onto the signal processing device 3. As another example, the signal processing program may be downloaded onto the signal processing device 3 via a data communication line such as the Internet.

The heartbeat data acquisition module 300 acquires heartbeat data indicating a heartbeat of the person to be measured in each of a plurality of time windows based on the Doppler signal. The heartbeat data acquisition module 300 includes a heartbeat data cut-out module 30 and a filter module 31. The heartbeat data cut-out module 30 applies a time window to data on the Doppler signal to cut out a data portion (heartbeat data) of the time window.

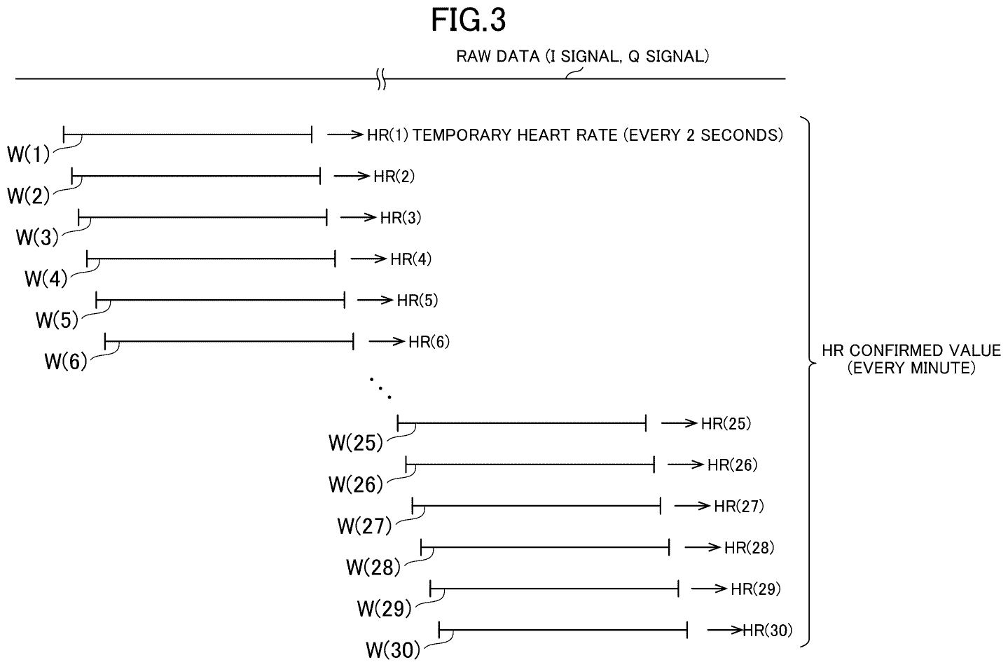

FIG. 3 is a diagram for illustrating processing performed by the heartbeat data cut-out module 30. As illustrated in the figure, a predetermined number of time windows (in this case, time windows W(1) to W(30)) are applied to data on the I signal and the Q signal per predetermined time period (in this case, for example, 1 minute), and a predetermined number (in this case, for example, 30) of pieces of heartbeat data are cut out. The time window has a fixed width (in this case, for example, 256 ms). A start timing of each time window is shifted by a predetermined time period (in this case, for example, 2 seconds).

As described later, a temporary heart rate HR(i) is generated from a time window W(i) (i=1 to 30). In addition, a heart rate confirmed value HR is generated based on the temporary heart rates HR(1) to HR(30). That is, the heart rate confirmed value HR is generated every minute in this case.

The heartbeat data (data on the I signal and the Q signal) of each time window W(i) is input to the filter module 31, and noise is removed therefrom. In this case, the filter module 31 includes a first filter module 31a and a second filter module 31b. The first filter module 31a may be formed of, for example, various band-pass filters, and extracts a frequency component corresponding to the heartbeat included in the heartbeat data.

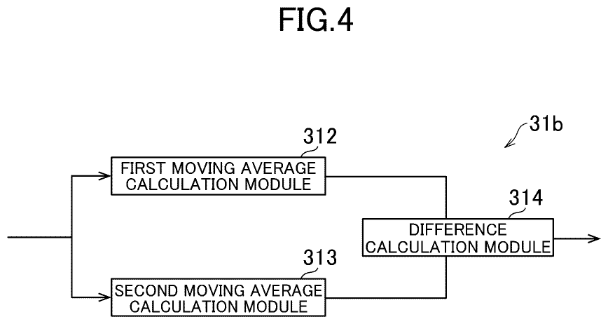

The second filter module 31b further removes a large trend derived from the noise from respiration or the like from output of the first filter module 31a. For example, as illustrated in FIG. 4, the second filter module 31b includes a first moving average calculation module 312, a second moving average calculation module 313, and a difference calculation module 314. The first moving average calculation module 312 calculates a moving average for a relatively short time period (in this case, for example, 0.1 second) in order to facilitate understanding of the signal component derived from the heartbeat. Meanwhile, the second moving average calculation module 313 calculates a moving average for a relatively long time period (in this case, for example, 1 second) in order to capture a signal component derived from respiration or the like. Then, the difference calculation module 314 subtracts the output of the second moving average calculation module 313 from the output of the first moving average calculation module 312. This enables a large trend derived from respiration or the like to be removed.

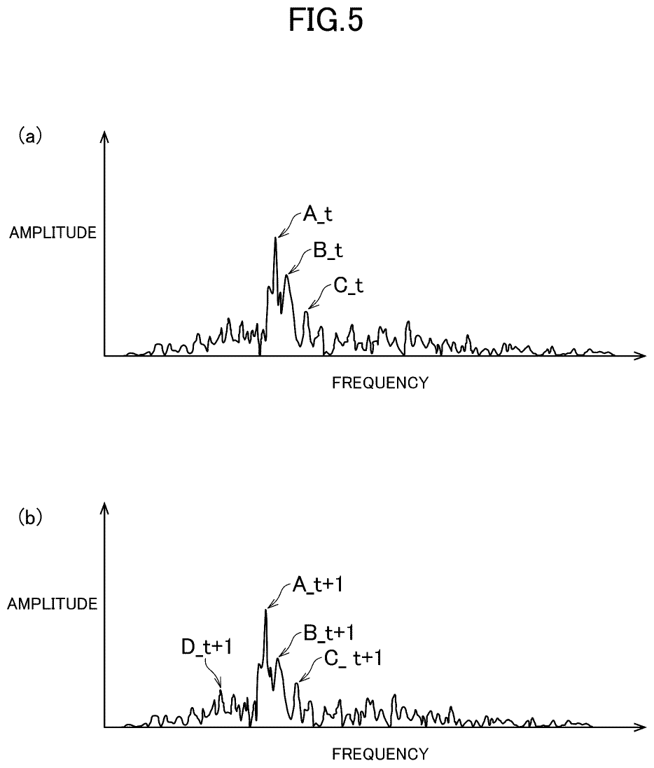

The heartbeat data (real number) from which the noise has been removed as described above is input to the FFT module 32. The FFT module 32 converts respective pieces of the heartbeat data into frequency spectra. FIGS. 5 are graphs for showing output of the FFT module 32. FIG. 5(a) shows a frequency spectrum at a time “t”, and FIG. 5(b) shows a frequency spectrum at a time t+1. As shown in those figures, the frequency spectrum output from the FFT module 32 generally includes peaks of various sizes.

The peak identification module 33 identifies peaks included in the frequency spectrum of each time window. Information on peaks satisfying a predetermined condition among the peaks identified by the peak identification module 33 is stored in the peak storage unit 35 by the peak tracking module 34. The data to be stored in this case includes a frequency and an amplitude of each peak. In order to evaluate a reliability level of the peak as described later, the peak identification module 33 selects a peak having the second largest amplitude in each time window, and stores an amplitude value of the peak in the peak storage unit 35.

The peak tracking module 34 determines whether or not peaks included in the frequency spectrum of each time window include a peak (corresponding peak) corresponding to a peak stored immediately before in the peak storage unit 35, that is, a peak extracted from the frequency spectrum relating to the immediately preceding time window. For example, when a peak is included within a predetermined width (for example, ±0.083 Hz) before and after the frequency of the peak stored immediately before, the included peak is determined as the “corresponding peak.” Then, data on such a corresponding peak is stored in the peak storage unit 35. In the example of FIGS. 5, peaks A_t, B_t, and C_t are present in descending order of the amplitude at the time “t” (FIG. 5(a)). At the time t+1 as well, a peak A_t+1 corresponding to the peak A_t, a peak B_t+1 corresponding to the peak B_t, and a peak C_t+1 corresponding to the peak C_t are present (FIG. 5(b)). That is, a frequency difference between the peak A t and the peak A_t+1, a frequency difference between the peak B_t and the peak B_t+1, and a frequency difference between the peak C_t and the peak C_t+1 are all equal to or smaller than a predetermined value.

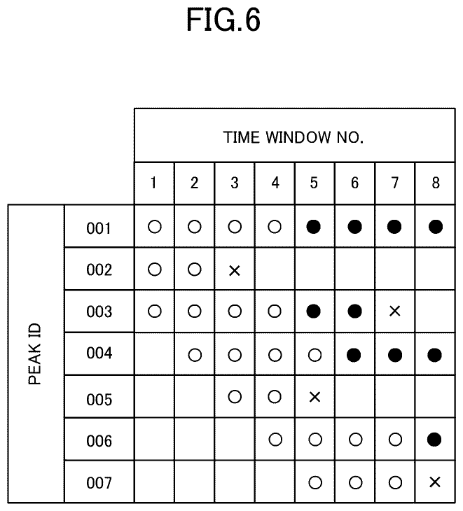

FIG. 6 shows an example of stored information of the peak storage unit 35. As shown in the figure, in a time window No. 1, data having peak IDs of 001 to 003 is stored in the peak storage unit 35. That is, at a start of an operation of the peak identification module 33, data on a predetermined number (in this case, three) of peaks from a frequency spectrum relating to the time window No. 1 in descending order of the amplitude is stored in the peak storage unit 35. In the figure, presence of the three peaks is indicated by “o”.

From a frequency spectrum relating to a time window No. 2, peaks corresponding to the peak IDs of 001 to 003 are identified, and data (frequency and amplitude) on the peaks is stored in the peak storage unit 35. In addition, a peak (additional peak) having the maximum amplitude among peaks to which no peak IDs have not yet been assigned is selected from the frequency spectrum relating to the time window No. 2, and the frequency and the amplitude of the additional peak are also stored in the peak storage unit 35. A unique peak ID of 004 is assigned to the additional peak. In the example of FIGS. 5, a peak D_t+1 corresponds to the additional peak.

From a frequency spectrum relating to a time window No. 3, the corresponding peaks have been extracted for the peak IDs of 001, 003, and 004, but no corresponding peak for the peak ID of 002 is present. The fact that no corresponding peak for the peak ID of 002 is present is indicated by “x” in the figure. An additional peak is selected from the frequency spectrum relating to the time window No. 3 as well, and the frequency and the amplitude of the additional peak are stored in peak storage unit 35. A peak ID of 005 is assigned to the additional peak.

From a frequency spectrum relating to a time window No. 4, the corresponding peaks for the peak IDs of 001 and 003 to 005 are extracted, and a peak having a peak ID of 006 is added.

From a frequency spectrum relating to a time window No. 5, the corresponding peaks for the peak IDs of 001, 003, 004, and 006 are extracted, and a peak having a peak ID of 007 is added. At this time, for the peak IDs of 001 and 003, the corresponding peaks are extracted in five consecutive time windows, and hence those peaks are regarded as a “valid peak.” This is indicated by “⋅” in FIG. 6. That is, the peak storage unit 35 stores, in association with each peak ID, whether the peak ID is a “temporary peak” (“o” in the figure) before being promoted to a “valid peak,” whether the peak ID is a “lost peak” (x in the figure) that has already vanished without the presence of the corresponding peak, or whether the peak ID is a “valid peak.” In this case, a peak is regarded as a “valid peak” when corresponding peaks therefor are present in five consecutive time windows, but may be regarded as a “valid peak” when corresponding peaks therefor are present in any number, which is three or more, of consecutive time windows.

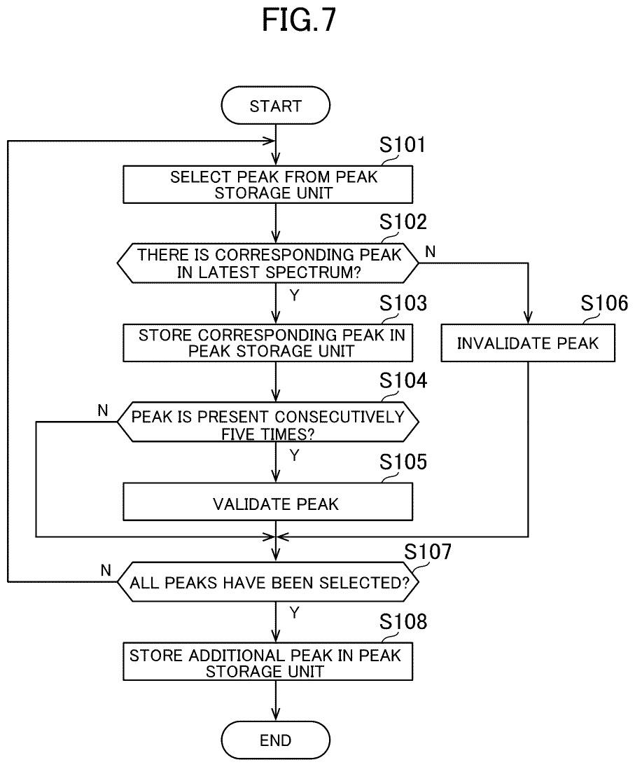

FIG. 7 is a flow chart for illustrating processing performed by the peak tracking module 34. The processing illustrated in the figure is executed on a frequency spectrum of each time window. First, the peak tracking module 34 selects one of the temporary peaks and the valid peaks stored in the peak storage unit 35 (Step S101). Subsequently, it is determined whether or not there is a corresponding peak for the peak selected in Step S101 among the peaks included in the frequency spectrum of the latest time window (Step S102). When there is a corresponding peak, data on the corresponding peak is stored in the peak storage unit 35 (Step S103). When corresponding peaks are present in a predetermined number (for example, five) of consecutive time windows (Step S104), the fact that the peaks are a “valid peak” is stored in association with the peak IDs of the peaks. Meanwhile, when it is determined in Step S102 that no corresponding peak is present, the fact that the peak selected in Step S101 is a “lost peak” is stored in association with the peak ID of the peak.

Then, the processing steps of from Step S102 to Step S106 are repeated for all the temporary peaks and all the valid peaks stored in the peak storage unit 35 (Step S101 and Step S107). When the processing steps of from Step S102 to Step S106 have been executed on all the temporary peaks and all the valid peaks (Step S107), an additional peak is selected from the latest frequency spectrum, and data on the additional peak is stored in the peak storage unit 35. At this time, a new peak ID is assigned to the additional peak, and the fact that the additional peak is a “temporary peak” is associated with the peak ID.

In this embodiment, when all the values of the frequencies of the peaks relating to the temporally adjacent time windows are within a predetermined range (for example, ±0.083 Hz) of each other in a predetermined number (in this case, five) of consecutive time windows, it is determined that the corresponding peaks are present, and the peaks are handled as a “valid peak.” As described later, in this embodiment, the heart rate of the person to be measured is calculated based on only the frequency of the “valid peak,” and hence a more reliable heart rate can be obtained.

The peak tracking module 34 may determine that corresponding peaks are present when values of frequencies of all the peaks relating to a predetermined number of consecutive time windows are within a predetermined range. That is, when a difference between the maximum value and the minimum value in frequencies of peaks relating to a predetermined number of consecutive time windows is equal to or smaller than a predetermined value, the peaks are determined to correspond to each other, and may be handled as a “valid peak.”

When data on the valid peak is stored in the peak storage unit 35 as described above, the heart rate generation module 301 generates a heart rate of the person to be measured based on the data. The heart rate generation module 301 includes a temporary heart rate calculation module 36, a temporary heart rate storage unit 37, a second reliability level determination module 38, and a heart rate confirmed value calculation module 39.

The temporary heart rate calculation module 36 includes a first reliability level determination module 36a. The first reliability level determination module 36a calculates a first reliability level for each valid peak of each time window stored in the peak storage unit 35. The first reliability level is an average value of relative amplitudes in the latest predetermined number (for example, five) of time windows in this case. The relative amplitude is a value obtained by dividing the amplitude of the valid peak by the amplitude of a peak having the maximum amplitude in each time window. The temporary heart rate calculation module 36 selects the valid peak having the highest first reliability level, and obtains a heart rate (BPM) from an inverse of the frequency of the peak. Then, the heart rate is stored in the temporary heart rate storage unit 37 as a temporary heart rate. The amplitude of the selected valid peak is also stored in the temporary heart rate storage unit 37. In addition, the second largest amplitude in each time window is stored in the temporary heart rate storage unit 37 as a second amplitude.

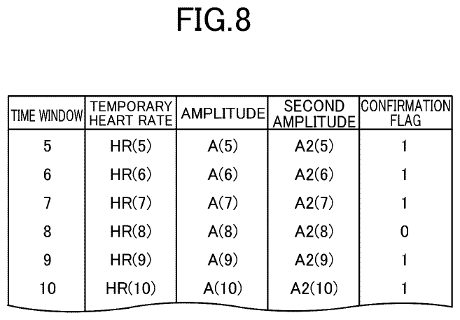

FIG. 8 is a table for schematically showing stored information of the temporary heart rate storage unit 37. As shown in the figure, the temporary heart rate storage unit 37 stores, for each time window, the temporary heart rate HR(i), an amplitude A(i) of the valid peak corresponding to the temporary heart rate, a second amplitude A2(i), and a confirmation flag.

An initial value of the confirmation flag is 0 (unconfirmed). The temporary heart rate storage unit 37 stores temporary heart rates corresponding to a determination cycle (in this case, one minute) for the heart rate. For example, when the time windows are set to be shifted from each other by two seconds with the determination cycle for the heart rate being one minute, 30 temporary heart rates are stored.

The second reliability level determination module 38 again determines the reliability level for the temporary heart rate of each time window stored in the temporary heart rate storage unit 37. Specifically, the second reliability level determination module 38 includes an amplitude ratio calculation module 38a, and the amplitude ratio calculation module 38a calculates an amplitude ratio by dividing the amplitude A(i) of the peak corresponding to the temporary heart rate for each time window by the second amplitude A2(i). When the amplitude ratio is equal to or larger than a predetermined threshold value (for example, 1.5) that is larger than 1, the second reliability level determination module 38 changes the value of the confirmation flag to 1. Thus, the temporary heart rate is handled as a confirmed value. Only when the amplitude ratio is equal to or larger than a predetermined threshold value that is larger than 1, the temporary heart rate is handled as the confirmed value, and hence it is possible to (1) prevent the heart rate from being calculated through use of a valid peak that does not have the maximum amplitude and (2) prevent the heart rate from being calculated through use of a valid peak that has a small amplitude ratio to the second largest peak. Accordingly, reliability of the finally calculated heart rate can be improved. The second reliability level determination module 38 may impose still another condition in order to change the confirmation flag to 1. For example, an additional condition that the amplitude of the valid peak exceeds a predetermined threshold value may be imposed.

The heart rate confirmed value calculation module 39 selects only the temporary heart rates HR(i) stored in the temporary heart rate storage unit 37 that have the confirmation flag set to 1, and calculates an average value thereof. Then, the average value is output as a heart rate confirmed value. At this time, when the confirmation flag is not set to 1 for a predetermined number (for example, 15) or more of temporary heart rates among the temporary heart rates calculated within the determination period, the heart rate confirmed value may not be output. This eliminates a probability of outputting a heart rate having a low reliability level.

According to the heart rate detection system 1 described above, peaks are identified from the frequency spectrum of each time window, and only when the peak is maintained over a predetermined number of consecutive time windows, the peak is handled as the valid peak to calculate the heart rate based on the valid peak, thereby being able to improve the reliability level of the heart rate.

Further, the ratio between the amplitude of the valid peak in each time window and the second amplitude (amplitude of the peak having the second largest amplitude) is calculated, and the reliability level of the temporary heart rate calculated from the valid peak is evaluated based on the amplitude ratio, thereby being able to further improve the reliability level of the heart rate.

### REFERENCE SIGNS LIST

1 heart rate detection system, 2 Doppler sensor, 3 signal processing device, 30 heartbeat data cut-out module, 31 filter module, 31a first filter module, 31b second filter module, 32 FFT module, 33 peak identification module, 34 peak tracking module, 35 peak storage unit, 36 temporary heart rate calculation module, 36a first reliability level determination module, 37 temporary heart rate storage unit, 38 second reliability level determination module, 38a amplitude ratio calculation module, 39 heart rate confirmed value calculation module, 300 heartbeat data acquisition module, 301 heart rate generation module, 312 first moving average calculation module, 313 second moving average calculation module, 314 difference calculation module.

The invention claimed is:

1. A heart rate detection system, comprising:
acquisition means for acquiring heartbeat data indicating a heartbeat of a person to be measured in each of a plurality of time windows based on a Doppler signal;
conversion means for converting respective pieces of the heartbeat data into frequency spectra;
peak identification means for identifying peaks included in each of the frequency spectra;
peak tracking means for determining whether corresponding peaks are present in the frequency spectra relating to a predetermined number of consecutive time windows, the predetermined number being three or more; and
heart rate generation means for generating a heart rate of the person to be measured based on a result of the determination.

2. The heart rate detection system according to claim 1, wherein the heart rate generation means is configured to generate, when the corresponding peaks are present in the frequency spectra relating to the predetermined number of consecutive time windows, the heart rate of the person to be measured based on at least some of the corresponding peaks.

3. The heart rate detection system according to claim 1, wherein the peak tracking means is configured to determine that the corresponding peaks are present when values of frequencies of peaks relating to temporally adjacent time windows are within a predetermined range.

4. The heart rate detection system according to claim 1, wherein the peak tracking means is configured to determine that the corresponding peaks are present when all values of frequencies of peaks relating to the predetermined number of consecutive time windows are within a predetermined range.

5. The heart rate detection system according to claim 1, further comprising amplitude ratio calculation means for calculating an amplitude ratio of a peak included in each of the frequency spectra to another peak,
wherein the heart rate generation means is configured to generate the heart rate of the person to be measured further based on the amplitude ratio.

6. The heart rate detection system according to claim 5, wherein the heart rate generation means is configured to generate the heart rate of the person to be measured based on only a peak the amplitude ratio of which is equal to or larger than a predetermined value.

7. The heart rate detection system according to claim 1,
wherein the heart rate generation means includes means for calculating temporary heart rates corresponding to the respective time windows, and
wherein the heart rate generation means is configured to avoid generating the heart rate of the person to be measured when the number of temporary heart rates that satisfy a predetermined criterion among the temporary heart rates calculated during a predetermined period is smaller than a predetermined number.

8. A heart rate detection method, comprising the steps of:
acquiring heartbeat data indicating a heartbeat of a person to be measured in each of a plurality of time windows based on a Doppler signal;
converting respective pieces of the heartbeat data into frequency spectra;
identifying peaks included in each of the frequency spectra;
determining whether corresponding peaks are present in the frequency spectra relating to a predetermined number of consecutive time windows, the predetermined number being three or more; and
generating a heart rate of the person to be measured based on a result of the determination.

9. A non-transitory computer readable information storage medium storing a program for causing a computer to execute:
acquiring heartbeat data indicating a heartbeat of a person to be measured in each of a plurality of time windows based on a Doppler signal;
converting respective pieces of the heartbeat data into frequency spectra;
identifying peaks included in each of the frequency spectra;
determining whether corresponding peaks are present in the frequency spectra relating to a predetermined number of consecutive time windows, the predetermined number being three or more; and
generating a heart rate of the person to be measured based on a result of the determination.
